(19) United States
(12) Patent Application Publication
(10) Pub. No.: US 2026/0014070 A1
(43) Pub. Date: Jan. 15, 2026

(54) COSMETIC COMPOSITION

(71) Applicants: Claudie BELLOUARD, New York, NY (US); Marta Alexandra de Sousa Oliveira MAIA, New York, NY (US); Enrica PERROTTA, New York, NY (US); Aurélien SAINT-PAUL, New York, NY (US); Lauren TIMONET, New York, NY (US); Dominique VERNAZ, New York, NY (US); Mark COOPER, New York, NY (US); Delphine LEQUINTREC, New York, NY (US); Karelle MESSNER, New York, NY (US); Veronique SCHWARTZ, New York, NY (US); Eva VIÑAS SANZ, New York, NY (US); Coty Inc., New York, NY (US); Karelle Messner, New York, NY (US)

(72) Inventors: Shimei Fan, New York, NY (US); Olivier Doucet, New York, NY (US); Stacey House, New York, NY (US); David Joyal, New York, NY (US)

(21) Appl. No.: 18/994,988

(22) PCT Filed: Jul. 19, 2023
(86) PCT No.: PCT/US2023/070528

§ 371 Date: Jan. 15, 2025

Related U.S. Application Data
(60) Provisional application No. 63/368,855, filed on Jul. 19, 2022.
(60) Provisional application No. 63/500,382, filed on May 5, 2023.
(60) Provisional application No. 63/500,388, filed on May 5, 2023.

(51) Int. Cl. A61K 8/9789; A61K 8/04; A61K 8/9783; A61Q 15/00

(52) U.S. Cl. CPC A61K 8/9789; A61K 8/046; A61K 8/9783; A61Q 15/00

(57) ABSTRACT

Various aspects disclosed relate to a cosmetic composition and methods associated comprising an extract solution comprising Nymphaea coerulea flower extract and Nelumbo nucifera flower extract.

### CROSS-REFERENCE TO RELATED APPLICATIONS

This application claims the benefit of priority under 35 U.S.C. § 119(e) of U. [symbol] Provisional Patent Application Ser. Nos. 63/368,855 filed Jul. 19, 2022; 63/500,382 filed [symbol] May 5, 2023; and 63/500,388 filed May 5, 2023, the disclosures of which are incorporated [symbol] herein in their entirety by reference.

### BACKGROUND

With conventional cosmetic products. a user typically must choose between [symbol] product that is easily removable and a product that remains on the skin for long periods of tim [symbol] (e.g., an entire day). Although easily-removable products can be rapidly removed by, f [symbol] example, washing with soap and water, a user may find themselves repeatedly reapplying t [symbol] product throughout the day, as the product may also be inadvertently removed by sweat a [symbol] contact with clothing or the like. On the other hand, typical long-lasting cosmetic products a [symbol] less subject to inadvertent removal, but may be difficult to wash off and may require the use [symbol] organic solvents or other harsh cosmetic removers. Accordingly, there is a continuing need [symbol] the art for cosmetic compositions that remain on the skin for extended periods of time, whi [symbol] being easily-removable by washing with soap and water. Transfer-resistant cosmetics typical [symbol] employ a film forming polymer to provide a long-wearing film of the skin, lips, hair or lash [symbol] and to aid in spreading and adhering the formulation to the surface. Such film forming polyme [symbol] may include polymers such as organosiloxanes, including polydimethylsiloxane (PDMS. [symbol] Dimethicone), utilized due to their excellent spreading properties and biological inertnes [symbol] More recently, the properties of silicone polymers have been modified by copolymerizati [symbol] with other polymers, such as polyurethanes, ethylenically unsaturated monomers or polyme [symbol] thereof, and the like. For example, in U.S. Patent Pub. 2008/0019932, the disclosure of whi [symbol] is hereby incorporated by reference, Revlon describes color cosmetic compositions comprisi [symbol] “at least one silicone film forming polymer, at least one pigment, and at least one dispersa [symbol] that aids in dispersion of the pigment and silicone film forming polymer in the composition.

[symbol]

Aesthetic conditions of the skin are frequently associated with natural [symbol] environmental causes, such as the process of aging (i.e., intrinsic aging), exposure to the su [symbol] or the resulting ultraviolet radiation therefrom (i.e., extrinsic aging), and the like. Other facto [symbol] such as improper care and/or diet, stress, nutritional deficiencies, repeated facial moveme [symbol] and genetic predisposition also contribute to the development of adverse skin conditions, such as fine lines, frown lines, folds, furrows and wrinkles.

Skin aging and wrinkling is a multifactor process. The formation of lines and wrinkles can be attributed to both extrinsic and intrinsic causes. Extrinsic causes include exposure to ultraviolet radiation and unquenched reactive oxygen species, smoke and other pollutants. Intrinsic factors include degradation of the skin matrix, loss of volume (fat and bone) and muscle tone.

With each smile, squint, laugh, or frown, the contraction of facial muscles causes dynamic wrinkles to appear on the face. In chronologically-younger skin, these lines dissipate quickly and are not prominent. However, with age, the lines become deeper and appear more noticeable. Most commonly, they are manifested as deep furrows between the eyebrows (“frown lines”), at the sides of the eyes (“crow's feet”), and across the forehead (“glabellar lines”).

The biochemistry and biomechanics of skeletal muscle contraction are well-characterized. Contraction is initiated by an electric signal (action potential) transmitted by the motor neuron. At the neuromuscular junction, the action potential causes vesicles to release the neurotransmitter acetylcholine (“Ach”) which, in turn, diffuses across the synapse between the nerve ending and the sarcolemma, the membrane of muscle fibers. ACh binds to ACh receptors, triggering the action potential to spread, and causing a release of calcium ions.

Muscle groups differ in their sensitivity to muscle relaxants. Muscles in the periorbital and glabellar areas are particularly sensitive to ACh and thus susceptible to effective treatment by agents that block Ach release. See, e.g., the review article published by L. Vimal and O. Oladapo, “Pharmacology of Non-Depolarising Muscle Relaxants” in Issue 5, Article 7 of Update in Anaesthesia, the electronic educational journal of World Anaesthesia and the World Federation of Societies of Anaesthesiologists. http://www.nda.ox.ac.uk/wfsa/html/u05/u05-012.htm accessed Jun. 20, 2007.

Botulinum toxin, (also known by the trade name, Botox®, Allergen, Irvine, Calif.), is currently in vogue for treating wrinkles and fine lines, but was initially used to treat spasms. This toxin acts on states of muscular spasticity by specifically inhibiting neurotransmission in nerve cells, thereby causing contracted muscles to relax (e.g., A. Blitzer et al., 1993, Arch. Otolaryngol. Head Neck Surg., 119:118-122; U.S. Pat. No. 6,344,461 B1 to L. Breton et al.). Botulinum toxin also can act on wrinkles of the glabella (wrinkles between the eyebrows) when injected subcutaneously. (See, J. D. Carruthers, 1992, J. Dermatol. Surg. Oncol., 18:17-21; U.S. Pat. No. 6,344,461 B1 to L. Breton et al.). However, the full extent of adverse effects related to long-term use of botulinum toxin and products or treatments containing this material are still not fully established. Botulinum toxin treatment has been associated with a number of side effects including, transient fatigue, dysphagia, neck weakness, hoarseness, and localized pain. In addition, many patients who preliminarily respond to botulinum toxin subsequently become non-responsive to treatment. (See, e.g., published U.S. Patent Application No. US2002/00812914 to Hawrot).

New ingredients for use as effective, active agents in compositions and formulations for application, preferably topical application, to the skin are advantageous to the industry and the consumer for a variety of uses in the areas of skin care treatment and therapy, and personal use products. Nelupure (INCI: Propanediol (and) Glycerin (and) Nymphaea caerulea Flower Extract (and) Nelumbo nucifera Flower Extract, used interchangeably throughout) is a blended extract from two types of lotus flower, the Egyptian Lotus (Nymphaea caerulea) and the Sacred Lotus (Nelumbo nucifera). It has been speculated that Nelupure (INCI: Propanediol (and) Glycerin (and) Nymphaea caerulea Flower Extract (and) Nelumbo nucifera Flower Extract) acts via leptin-receptor inhibition to regulate the sebaceous glands, preventing the excess of sebum production and inflammatory processes that cause skin blemishes.

Unwind Sacred Lotus, marketed by the Naolys company, is a material consisting of active plant cells from Nelumbo nucifera whose action has been described as “modulating the skin's neuro-system, Sacred Lotus cells have a relaxing effect that helps to restore the skin's original balance.” https://www.unifect.com/products/unwind-sacred-lotus/

### BRIEF DESCRIPTION OF THE FIGURES

The drawings illustrate generally, by way of example, but not by way of limitation, various aspects of the present disclosure.

FIG. 1 is a flow chart of a protocol used in conjunction with Example 3.

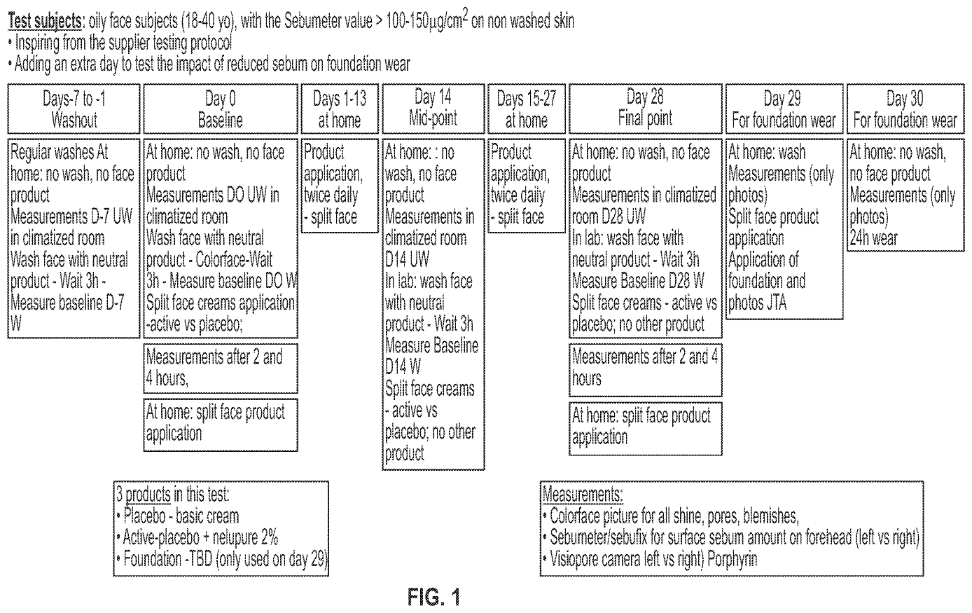

### SUMMARY OF THE DISCLOSURE

Various aspects disclosed relate to a cosmetic composition comprising any of the components disclosed herein. The present disclosure provides compositions and methods comprising lotus plant derived constituents newly found to be effective to treat, including prevent, reduce, ameliorate, and/or eliminate, signs and results of dermatological aging of skin, especially wrinkles and fine lines, and/or to improve the aesthetic appearance of skin. Further these constituents are provided for use in compositions and methods to treat, including prevent, ameliorate, and/or reduce, hyperhidrosis (or sweating or perspiration) and its associated effects, such as malodor. More specifically, the active constituents (also referred to as active agents, components, ingredients, reagents, or compounds herein) are extracts of the lotus flower and/or blends of extracts of various species of the lotus family such as but not limited to Nelupure (INCI: Propanediol (and) Glycerin (and) Nymphaea caerulea Flower Extract (and) Nelumbo nucifera Flower Extract), obtained from plants and the like, of the Nelumbo family. These plant materials have been newly found to provide treatment for wrinkles, fine lines and other signs of dermatological aging (i.e., intrinsic aging) or sunlight exposure of the skin (i.e., extrinsic aging), as well for use in the treatment of hyperhidrosis, by virtue of their effect at the neuromuscular junction (NMJ) of muscle and nerve cells and tissue, and at the fibers of the sweat glands to inhibit acetylcholine release, e.g., at the NMJ, thereby relaxing or decontracting muscles, e.g., facial expression muscles.

It has been found that the incorporation of an active ingredient capable of reducing integument sebum production may yield a cosmetic composition capable of remaining on the skin for extended periods of time. The present disclosure provides a longwear composition that includes an active component. The active component can include a material that inhibits integument sebum production. In one aspect, the active ingredient downregulates integument sebum production via leptin receptor inhibition. In another aspect, the active ingredient downregulates integument sebum production via competitive inhibition of cell surface leptin receptor activity. In another aspect, the active ingredient downregulates integument sebum production via nicotinic acetylcholine receptor (nAchR) downregulation. In another aspect, the active ingredient downregulates integument sebum production via competitive inhibition of nAchR activity. In another aspect, the active ingredient inhibits acetylcholinesterase activity; generation; and or stability. In another aspect, the active ingredient inhibits acetylcholine release.

In certain aspects, the skin in need of the disclosed composition(s), may be skin having excess sebum. In a further aspect of a method for treating skin with excess sebum, the skin benefit may selected from the group consisting of: restoration of a matte finish to the skin; an evening of skin type; reduction in oily/greasy feel to skin and/or hair; reduction in the incidence of dandruff; reduction in the incidence of blocked/clogged pores; reduction in the incidence of comedones; reduction in the incidence of acne lesions; reduction in the area over expressing or over-producing lipids; reduction in thickness of skin affected by over-production of lipids; and/or combinations thereof.

It is another aspect of the present disclosure to provide compositions, formulations and methods containing materials newly determined to be useful in the treatment of wrinkles, fine lines, folds, furrows and other signs of aging skin, in addition to treating, preventing or reducing hyperhidrosis. These materials exert their effectiveness according to this disclosure by working at the neuromuscular junctions (NMJ), such as in the muscle and nerve cell or tissue layer of a site of application, e.g., skin of the face, neck, arms, feet, or hands, or in the dermal layer of the skin where sweat glands are located. More specifically, the extracts from the flower of one or more species of the lotus flower constituents block or inhibit the release of acetylcholine at the NMJ of skeletal muscle, which, in turn, results in the relaxation or decontraction of contracted muscles. Contracted muscles are associated with the formation of fine lines, wrinkling and the like.

It is to be understood that, as used herein, the terms treating and treatment include and encompass preventing, reducing, ameliorating, improving, alleviating, and/or eliminating the dermatological effects of aging and sun exposure, with particular regard to wrinkles, fine lines, folds, furrows, creases of the skin, and the like. The present disclosure further encompasses the treatment, as defined above, of “marionette” lines that run on either side of the mouth, as well as lines on the forehead, and the perpendicular lines between the brows. The present compositions and methods are also suitable for use in treating, as defined above, dermatological conditions of the skin in numerous areas of the body, including, without limitation, the face, forehead, neck, arms, hands, legs, knees, feet, chest, back, groin, buttocks, and the like.

### DETAILED DESCRIPTION OF THE DISCLOSURE

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

As used herein, the term “composition” includes a composition intended for application to a body surface, such as for example, skin or hair. The term “composition” may also include cleaning compositions, such as fabric care composition or home care compositions, including air care compositions (e.g., air fresheners), for use on clothing or other substrates such as hard surfaces (e.g., dishes, floors, countertops). Additional non-limiting examples of “composition” may also include facial or body powder, deodorant, foundation, body/facial oil, mousse, creams (e.g., cold creams), waxes, sunscreens and blocks, bath and shower gels, lip balms, self-tanning compositions, masks and patches.

As used herein, the term “consumer” means both the user of the composition and the observer nearby or around the user.

It is understood that the test methods that are disclosed in the Test Methods Section of the present application may be used to determine the respective values of the parameters of Applicants' disclosures as described and claimed herein.

In all aspects of the present disclosure, all percentages are by weight of the total composition, as evident by the context, unless specifically stated otherwise. All ratios are weight ratios, unless specifically stated otherwise, and all measurements are made at 25° C., unless otherwise designated

A pH of the composition can be in a range of from about 4 to about 8, about 5to about 7, less than, equal to, or greater than about 4, 5, 6, 7, or about 8. The ethanol concentration in any of the compositions described herein can be less than about 78 wt % ethanol, less than about 70 wt % ethanol, less than about 60 wt % ethanol, less than about 50wt % ethanol, less than about 40 wt % ethanol, less than about 30 wt % ethanol, less than about 20 wt % ethanol, less than about 10 wt % ethanol, or free of ethanol.

### Epidermis and Dermis

Human skin comprises two major compartments: a superficial outer compartment, the epidermis, and a deeper compartment, the dermis. The outermost epidermal skin layers typically provide a certain degree of protection to the body, although fine lines and wrinkles can be readily visualized in this portion of the skin. The epidermis and dermis bear the brunt of harmful effects of photodamage. The natural human epidermis functions to provide the essential protectant role of the skin in the human body. The dermis, which provides a solid and nutritional support to the epidermis, comprises mainly fibroblasts and an extracellular matrix composed primarily of a substance that includes collagen, elastin and glycosaminoglycans (“GAGs”). In addition, the dermis contains leukocytes, mast cells, tissue macrophages, blood vessels, muscle cells and nerve fibers. The dermal muscles of the face are controlled by motor nerve afferences of the facial nerve. The interlobular septa of the hypoderm contain within them fibers that constitute a striated muscle tissue, i.e., the panniculus carnosus. It is known that a subpopulation of dermal fibroblasts, called myofibroblasts, has contractile characteristics similar to those of muscle cells and tissue.

### Facial Lines

Glabellar facial lines arise from the activity of the corrugator and orbicularis oculi muscles. These muscles move the brow medially, and the procerus and depressor supercilii pull the brow inferiorly. This creates a frown or “furrowed brow”. The location, size, and use of the muscles vary markedly among individuals. Lines induced by facial expression occur perpendicular to the direction of action of contracting facial muscles. An effective dose for facial lines is determined by gross observation of the patient's ability to activate the superficial muscles injected.

Lateral canthal lines arise largely from the activity of the orbicularis oculi muscles around the eye responsible for blinking and eyelid closure. Forceful contraction of the orbicularis oculi results in lateral and radially oriented folds (crow's feet lines) which originate from the lateral canthus. The distribution of these radial lines differs among patients. Forehead lines arise largely from the activity of the frontalis muscles. This muscle moves the brow superiorly, interacting with the procerus, orbicularis, corrugator, and depressor supercilli. Frontalis contraction causes brow elevation. The location, size, and use of the muscles vary markedly among individuals.

### Hyperseborrhoeic Skin

Some people exhibit hyperseborrhoeic skin characterized by an exaggerated secretion and excretion of sebum. Generally, individuals with hyperseborrhoeic skin exhibit sebum levels of greater than 200 μg/cm² measured on the forehead. This overproduction of sebum may lead to aesthetic problems, such as oily/shiny skin or hair, acne-prone skin, enlarged pores, thickened skin, or poorer hold of makeup. Additionally, the excess sebum may act as a catalyst for acne by clogging pores leading to the formation of comedones (“blackheads” or “whiteheads”), which, when left untreated, may become inflamed and progress into acne vulgaris. Additionally, odors may be emitted as a result of excess sebum accumulation, production, or excretion. The compositions described herein can provide a skin benefit in that they can prevent these undesirable effects caused by excess sebum production.

In a further aspect, the skin benefit may be selected from the group consisting of: reducing lipid production by sebaceous glands; reducing lipid synthesis in subcutaneous adipose tissue; reducing triglyceride synthesis; reducing fatty acid synthesis; preventing and/or improving conditions related to skin associated with inhibited lipolysis; preventing and/or improving conditions related to skin associated with nonselective tor partially selective PPAR-.gamma. stimulators/up regulators; preventing and/or improving conditions related to skin associated with nonselective or partially selective up regulation of adipogeneic genes typically up regulated by PPAR-. gamma. activation; preventing and/or improving conditions related to skin associated with nonselective or partially selective FAS stimulators/up regulators; preventing and/or improving conditions related to skin associated with nonselective or partially selective SCD-1 receptor stimulators/up regulators; preventing, ameliorating or treating oily skin; preventing, ameliorating or treating oily hair; preventing, ameliorating or treating oily scalp; preventing, ameliorating or treating enlarged pores; preventing, ameliorating, or treating acne-prone skin; preventing, ameliorating or treating body odors associated with excess sebum production; preventing, ameliorating or treating cellulite in adults; preventing, ameliorating or treating excess accumulation or production of subcutaneous fat in adults; improving skin texture associated with cellulite; and/or combinations thereof.

In certain aspects, the skin in need of the disclosed composition(s), may be skin having excess sebum. In a further aspect of a method for treating skin with excess sebum, the skin benefit may selected from the group consisting of: restoration of a matte finish to the skin; an evening of skin type; reduction in oily/greasy feel to skin and/or hair; reduction in the incidence of dandruff; reduction in the incidence of blocked/clogged pores; reduction in the incidence of comedones; reduction in the incidence of acne lesions; reduction in the area over expressing or over-producing lipids; reduction in thickness of skin affected by over-production of lipids; and/or combinations thereof.

According to various aspects of the present disclosure a cosmetic composition can include an active component.

### Acetylcholine and Acetylcholine Release Inhibitors

The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the bag 1 fibers of the muscle spindle fiber, by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephine. In most instances acetylcholine has an excitatory effect. However, acetylcholine is known to have inhibitory effects at some of the peripheral parasympathetic nerve endings, such as inhibition of heart rate by the vagal nerve.

The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since, the preganglionic neurons of both the sympathetic and parasympathetic nervous system are cholinergic, application of acetylcholine to the ganglia will excite both sympathetic and parasympathetic postganglionic neurons.

Acetylcholine activates two types of receptors, muscarinic and nicotinic receptors. The muscarinic receptors are found in all effector cells stimulated by the postganglionic, neurons of the parasympathetic nervous system as well as in those stimulated by the postganglionic cholinergic neurons of the sympathetic nervous system. The nicotinic receptors are found in the adrenal medulla, as well as within the autonomic ganglia, that is on the cell surface of the postganglionic neuron at the synapse between the preganglionic and postganglionic neurons of both the sympathetic and parasympathetic systems. Nicotinic receptors are also found in many nonautonomic nerve endings, for example in the membranes of skeletal muscle fibers at the neuromuscular junction.

Acetylcholine is released from cholinergic neurons when small, clear, intracellular vesicles fuse with the presynaptic neuronal cell membrane. A wide variety of non-neuronal secretory cells, such as, adrenal medulla (as well as the PC12 cell line) and pancreatic islet cells release catecholamines and parathyroid hormone, respectively, from large dense-core vesicles. The PC12 cell line is a clone of rat pheochromocytoma cells extensively used as a tissue culture model for studies of sympathoadrenal development.

A neuromuscular junction is formed in skeletal muscle by the proximity of axons to muscle cells. A signal transmitted through the nervous system results in an action potential at the terminal axon, with activation of ion channels and resulting release of the neurotransmitter acetylcholine from intraneuronal synaptic vesicles, for example at the motor endplate of the neuromuscular junction The acetylcholine crosses the extracellular space to bind with acetylcholine receptor proteins on the surface of the muscle end plate. Once sufficient binding has occurred, an action potential of the muscle cell causes specific membrane ion channel changes, resulting in muscle cell contraction. The acetylcholine is then released from the muscle cells and metabolized by cholinesterases in the extracellular space. The metabolites are recycled back into the terminal axon for reprocessing into further acetylcholine.

### Sebum and Sweat Reduction With Proposed Mechanism of Action (Leptin Receptor Downregulation/Acetylcholine Release Inhibition)

It has been found that the incorporation of an active ingredient capable of reducing integument sebum production may yield a cosmetic composition capable of remaining on the skin for extended periods of time. The present disclosure provides a cosmetic composition that includes an active component. The active component can include a material that inhibits integument sebum production. In one aspect, it has been proposed that the active ingredient downregulates integument sebum production via leptin receptor downregulation. In another aspect, the active ingredient downregulates integument sebum production via competitive inhibition of cell surface leptin receptor activity. In another aspect, the active ingredient inhibits acetylcholine release. The active does not downregulate sebum via ach receptor. The active was found to inhibit acetylcholine release.

### Mechanism of Action

In one aspect, the active component is a leptin receptor downregulator and/or acetylcholine release inhibitor. In one aspect, the leptin receptor downregulator and/or acetylcholine release inhibitor is an extract solution that can include nymphaea coerulea flower extract and nelumbo nucifera flower extract. In one aspect, the solution is available as a product under the tradename NELUPURE (INCI: PROPANEDIOL (AND) GLYCERIN (AND) NYMPHAEA CAERULEA FLOWER EXTRACT (AND) NELUMBO NUCIFERA FLOWER EXTRACT). Without intending to be bound to any theory, it is thought that the extract solution is useful in a cosmetic composition in part because it reduces the expression of the leptin cellular receptor in sebocytes, thus preventing the triggering of sebum production and inflammatory processes that cause skin blemishes; and/or inhibits acetylcholine release at the neuromuscular junction. It has been shown that leptin, a protein synthesized and secreted by different cell types in our body, including skin cells plays a role in the cellular mechanism that triggers sebum synthesis. The benefits of including the extract solution are at least somewhat unexpected because it may be an inhibitor of acetylcholine release. According to some aspects, the ability of the extract solution to function as a component of a cosmetic composition is determined by an assay for determining the levels of sebum; leptin cell surface receptors in sebocytes; and/or, acetylcholine release inhibition in a sample that has been contacted with the extract or a composition including the extract.

The ability of the extract solution of nymphaea coerulea flower extract and nelumbo nucifera flower extract to provide beneficial properties as an acetylcholine release inhibitor suggests that other natural extracts with acetylcholine release inhibition properties can also be used for the modalities described herein.

According to some aspects, the ability of the extract solution to function as a component of a cosmetic composition is determined by an assay for determining the levels of acetylcholine (e.g., an ELISA assay in a sample that has been contacted with the extract or a composition including the extract.

The present disclosure provides novel compositions and methods comprising components newly found to be effective to treat, including prevent, reduce, ameliorate, inhibit, alleviate, and/or eliminate signs and results relating to the dermatological aging of skin due to chronological and/or hormonal aging, or due or to photoexposure (e.g., to the sun, or ultraviolet radiation) of skin, especially wrinkles, creases, furrows, folds and fine lines; and/or to improve the aesthetic appearance of skin. These components are further provided for use in compositions and methods to treat, including prevent, ameliorate, inhibit and/or reduce, hyperhidrosis (sweating or perspiration) and its associated effects, such as malodor and unaesthetic appearance.

More specifically, the components (also referred to as active agents, constituents, ingredients, reagents, substances, or compounds herein) of this disclosure are extracts from the flower of one or more species of the lotus flowers, obtained from plants and the like, preferably of the Nelumbo family. These plant materials have been newly determined to be effective constituents in compositions and methods for treating, preventing, reducing, inhibiting, improving and/or eliminating wrinkles, fine lines, creases, folds, furrows, and other signs of dermatological aging due to chronological and/or hormonal aging, or due to sunlight exposure of the skin, as well for use in the treatment of hyperhidrosis.

According to the present disclosure, yet without wishing to be bound by theory, the extracts from the flower of one or more species of the lotus flower constituents exert their effects by their ability to inhibit, block, reduce, or prevent the release of acetylcholine at the neuromuscular junction (NMJ) of skeletal muscle cells and tissue, thereby relaxing muscles, for example, muscles associated with facial movement or expressions. Both nerve and muscle cells (skeletal muscle cells) are electrically excitable and the junctions between these types of cells are known as NMJ. It has been known for many years that the stimulation of a motor nerve innervating a skeletal muscle also causes the release of acetylcholine and that acetylcholine, in turn, stimulates the skeletal muscle to contract. Thus, acetylcholine has been identified as the neurotransmitter at the neuromuscular junction. (see, e.g., B. Alberts et al., 1989, Molecular Biology of the Cell, 2^{nd} Ed., Chapter 19, The Nervous System, Garland

Publishing, Inc., NY., pp. 1075 et seq.). Provided by the present disclosure from the flower of one or more species of the lotus flower constituents which are newly described herein to inhibit, reduce, block, or prevent the release of acetylcholine at the NMJ of the skeletal muscle and thus relax the muscles, preferably those associated with facial expression or movement, particularly when employed in the compositions and methods of the invention. By inhibiting acetylcholine release, compositions and methods comprising the extracts from the flower of one or more species of the lotus flower constituents as described herein can reduce, treat, and/or prevent excessive sweating or perspiration.

The ability of the extracts from the flower of one or more species of the lotus flower constituents to inhibit acetylcholine release by muscle cells results in a modulation of motor contraction so as to relax the muscle fibers in cutaneous or subcutaneous muscle and/or nerve tissue, thereby attenuating wrinkles, as well as fine lines, folds, furrows, and the like. By relaxing or preventing the contraction or hypercontraction of the cutaneous or subcutaneous muscle cells of areas such as the face, or hands, feet, etc., the extracts from the flower of one or more species of the lotus flowers and compositions containing extracts from the flower of one or more species of the lotus flowers can effectively smooth out the landscape of the skin in those areas where muscle contraction is involved in the formation of wrinkles and the like. Thus, if muscle cell contractility is associated with the formation of wrinkles, fine lines, etc., the relaxation or decontraction of the contractility of cutaneous or subcutaneous muscle tissue by the extracts from the flower of one or more species of the lotus flower actives in the compositions of this disclosure can serve to loosen or slacken the contracting muscle tissue and alleviate, reduce, ameliorate, inhibit, or eradicate the wrinkles, fine lines, etc. Contraction of cutaneous or subcutaneous muscle cells or tissue of skin can elicit wrinkling, fine lines, and the like, which constitute at least some of the visible dermatological effects of aging due to chronological and/or hormonal aging, and/or due to photoexposure, as described herein.

Thus, in one of its aspects, the present disclosure encompasses compositions, formulations and methods containing components, preferably, extracts from the flower of one or more species of the lotus flower constituents, newly determined to be useful in the treatment of wrinkles, fine lines, folds, furrows and other signs of aging and/or photoexposure of skin, in addition to treating, preventing or reducing hyperhidrosis. Skin in a variety of areas of the body is amenable for treatment and/or receipt of the compositions of the present disclosure the face, forehead, neck, arms, legs, hands, feet, torso (chest), back, and the like.

The plant-derived materials from the Lotus family exert their effectiveness according to this disclosure by preferably working at the neuromuscular junctions (NMJ), such as in and around muscle and nerve cells and tissue at a site of application, e.g., the skin of face, neck, arms, feet, hands, or chest, or in the dermis layer of the skin where sweat glands are located. Prior to the present materials in combination were not previously known or recognized to inhibit acetylcholine release at NMJ of skeletal muscles, thereby relaxing muscles associated with aesthetically displeasing wrinkles, frown lines, fine lines, folds, furrows, creases, neck bands, and the like, that can arise from aging due to chronological and/or hormonal aging, prolonged or overexposure of the skin to the sun, ultraviolet radiation associated with photoexposure, or over-exercised expression muscles.

In general, for the purposes of the present disclosure, a substance, such as extracts from the flower of one or more species of the lotus flower constituent of the described compositions, is recognized as being a muscle relaxant when it elicits a relaxation effect on contracted muscle cells or on tissue, such as cutaneous or subcutaneous muscle tissue, and/or exhibits an inhibitory effect on acetylcholine release at the NMJ. Contracted muscle cells or tissue is associated with formation of wrinkles, fine lines, etc. Relaxation or decontraction of contracted muscle, such as by the action of extracts from the flower of one or more species of the lotus flowers to inhibit or block acetylcholine release at the NMJ, serves to smooth out the landscape, or microrelief, of the skin, thereby effecting the prevention, amelioration, reduction, and/or eradication of wrinkling and fine line, etc. formation caused by contracted muscle tissue in skin.

For use in the compositions of this disclosure, the extracts from the flower of one or more species of the lotus flower extracts can be chemically synthesized. Alternatively, these plant alkaloids may be extracted from plants. When extracted, they may be in a pure form, a semi-purified form, or may be a component of an unpurified plant extract.

The extracts from the flower of one or more species of the lotus flower plant materials contained in the compositions of the present disclosure can be chemically synthesized at industrial scale in large amounts. Alternatively, the materials may be extracted from natural raw materials from plants. Any and all methods of preparation may be used, including the use of cultured plant seed cells, as disclosed in commonly owned patent application U.S. Ser. No. 10/040,242, which is hereby incorporated by reference herein. For example, unpurified plant extracts may be employed in the present disclosure on the solubility of the particular plant extract in water or in organic solvent, the extraction process may differ. Two methods for extracting the materials from raw plant materials include organic solvent extraction, and aqueous-organic solvent extraction, as described in U.S. Pat. No. 6,372,239 to Wu et al. The organic extraction method involves a step of continuous washing and extracting the plant material against a stream of organic solvent. Examples of organic solvents include, but are not limited to methanol, ethanol, dichloromethane, chloroform, xylene, and petroleum ether. Alternatively, the alkaloid can be partially purified or completely purified. Chemical synthesis of the alkaloid obviates the need for extraction and purification.

In accordance with this disclosure, the extracts from the flower of one or more species of the lotus flower constituents comprise compositions which include, without limitation, topically applied formulations, anti-oxidants, anti-inflammatoires, sunscreens, cosmetics, including make-ups, anti-aging formulations, e.g., creams for fine lines and/or wrinkles, topicals, skin penetration enhancers, antiperspirants, deodorants, and the like. Also in accordance with this disclosure from the flower of one or more species of the lotus flower constituents and additional ingredients comprising such compositions can be formulated in a variety of product forms. Preferably, the compositions are prepared in targeted delivery systems, e.g., transdermal, such as patches, and the like, particularly for topical administration.

The present disclosure encompasses compositions comprising one or more extracts from the flower of one or more species of the lotus flower constituents, preferably in a pharmaceutically-acceptable cosmetic or dermatological formulation which is suitable for contact with living animal tissue, including human tissue, and for topical administration, with virtually no adverse physiological effect, e.g., irritation, to the user. Thus, the inventive compositions are especially suitable for sensitive skin.

Compositions embraced by this disclosure can be provided in any cosmetically and/or dermatologically suitable form, preferably as a lotion, gel, cream, or incorporated into a transdermal patch, and also in an ointment or oil base, as well as in a sprayable liquid form. Other suitable cosmetic product forms for the compositions of this disclosure for example, an emulsion, a mousse, a lip balm, a lip gloss, a lotion, a mask, an ointment, a pomade, a solution, a serum, a spray, or a towelette. In addition, the compositions contemplated by this invention can include more compatible cosmetically acceptable adjuvants commonly used and known by the skilled practitioner, such as colorants, fragrances, emollients, humectants, preservatives, vitamins, chelators, thickeners, and the like, as well as other botanicals such as aloe, chamomile, and the like, and as further described below. The compositions are preferably applied topically once or twice daily. The daily application can be for periods of up to two weeks, four weeks, or more.

In one aspect encompassing sunscreen formulations, the compositions comprising extracts from the flower of one or more species of the lotus flower constituents can further include, without limitation, ingredients such as avobenzone, cinnamic acid derivatives (e.g., octylmethoxycinnamate), octyl salicylate, oxybenzone, titanium oxide, zinc oxide and mixtures or combinations thereof. Such formulations can also preferably include an alpha hydroxy acid, an oxa acid, an oxa diacid, and mixtures or combinations thereof.

The compositions of this disclosure can also be formulated into liposomes which can comprise other additives or substances, and/or which can be modified to more specifically reach or remain at a site following administration. Alternatively, the extracts from the flower of one or more species of the lotus flower-containing compositions can be injected subcutaneously (s.c.) or intradermally (i.d.) (for example, via microneedling optionally associated with a transdermal patch) at a site in need of wrinkle, fine line, etc. reduction, improvement, prevention, and/or elimination, resulting from inhibition of acetylcholine release at the NMJ and concomitant relaxation of contracted muscle.

Pharmaceutical or physiologically acceptable compositions suitable for use in the present disclosure include compositions in which the active constituents, ingredients, or materials are contained in an amount effective to achieve the intended purpose. By way of example, in the present compositions, extracts from the flower of one or more species of the lotus flower constituent is present in an amount of from about 0.0001 wt % to about 10 wt %, based on the total weight of the composition. More preferably, the present compositions include one or more extracts from the flower of one or more species of the lotus flower constituents in an amount from about 0.0005 wt % to about 5 wt %. Most preferably, the present compositions include one or more extracts from the flower of one or more species of the lotus flower constituents in an amount from about 0.001 wt % to about 2 wt % of the total composition.

The determination of an effective dose or amount is well within the capability of those skilled in the art. A therapeutically effective dose refers to that amount of active ingredient, for example, extracts from the flower of one or more species of the lotus flower constituent identified in accordance with the present disclosure, which, for instance, treats, prevents, ameliorates, reduces, or eliminates the condition, more specifically, wrinkles, fine lines, creases, and the like. The practitioner, who will consider the factors related to the individual requiring treatment, will determine the exact dosage. Dosage and administration are adjusted to provide sufficient levels of the active constituent or to maintain the desired effect. Factors which are typically considered include the severity of the individual's particular need, general health of the patient, age, weight, and gender of the individual, diet, time and frequency of administration, drug combination(s), reaction sensitivities, and tolerance/response to treatment. As a general guide, long-acting pharmaceutical compositions can be administered once daily, every 2 to 4 days, every week, or once every two weeks, depending on half-life and clearance rate of the particular formulation. Variations in these dosage levels can be adjusted using standard empirical routines for optimization, as is well understood in the art. Guidance as to particular dosages and methods of delivery is provided in the literature and is generally available to practitioners in the art. Those skilled in the art will employ different formulations depending upon the nature, e.g., structure, composition, of the active extracts from the flower of one or more species of the lotus flower constituent.

The compositions of the present disclosure yield improvements to the aesthetic appearance of the skin by treating at least one of the following: signs of dermatological aging, especially chronological, actinic or hormonal aging, or signs of extrinsic aging, such as sun exposure. In particular, improvements to the aesthetic appearance of skin include at least one of the following: makes facial lines appear less noticeable, makes facial lines and/or wrinkles feel plumped, improves appearance of suborbital lines and/or periorbital lines, improves appearance of crow's feet, reduces and/or diminishes the appearance of wrinkles, particularly facial wrinkles on the cheeks, forehead (e.g., perpendicular wrinkles between eyes, horizontal wrinkles above the eyes), and/or around the mouth, (e.g., marionette lines), and particularly deep wrinkles, folds, or creases, improves skin suppleness, reduces and/or eliminates fine and/or deep lines, folds and creases, and smoothes skin, e.g., to the extent that it reduces wrinkling/lines.

Embraced by the present disclosure are transdermal modes of delivery, such as patches and the like, with or without a suitable skin penetration enhancer. The methods and compositions embodied by the disclosure include a means by which the one or more extracts from the flower of one or more species of the lotus flower constituents can be effectively administered in a transdermal system. Frequently, compounds having poor topical absorption, or which are required at high dosage levels, are delivered transdermally. Accordingly, a transdermal means of delivering a composition or formulation (often with a skin penetration enhancer composition) to the skin is that of the transdermal patch or a similar device as known and described in the art. Examples of such devices are disclosed in U.S. Pat. Nos. 5,146,846, 5,223,262, 4,820,724, 4,379,454 and 4,956,171; such descriptions are not meant to be limiting. The transdermal mode of storing and delivering the compositions onto the skin and forming the active composition is convenient and well suited for the purposes of an aspect of the present disclosure a topical patch is used, the patch is applied to the desired area for extended period of time. Preferably, the extended period of time is greater than one hour; more preferably, the extended period of time is overnight, e.g., when the user is sleeping.

A particular aspect of the present disclosure is directed to the delivery of the

described compositions by the use of targeted delivery systems, for example, liposomes, microspheres (see, e.g., U.S. Pat. No. 5,770,222 to Unger et al.), transdermal patches, and the like, so that the extracts from the flower of one or more species of the lotus flower actives can more readily reach and affect the muscle layer of the area of application, e.g., face or neck, or the dermis layer of the skin, where sweat glands are located, e.g., underarm, foot, hand, etc.

Compositions comprising extracts from the flower of one or more species of the lotus flower constituents, including liposome formulations, can also be administered by direct injection subcutaneously or intradermally to more precisely deposit the active agents at sites which require muscle relaxation or decontraction via acetylcholine release inhibition.

Liposomes and delivery systems and vehicles involving liposomes are well-known in the art. In brief, liposomes are unilamellar or multilamellar lipid vesicles which entrap a significant fraction of aqueous solution. The vesicular microreservoirs of liposomes can contain a variety of water-soluble materials, which are suspended within the emulsion (e.g., reviewed in G. Gregorius (Ed.), 1991, Liposome Technology, Vols. I, II, III, CRC Press, Boca Raton, Fla.; Davis S. S. and Walker I. M., 1987, Methods in Enzymology, 149:51-64; Mayhew E. et al., 1987, Methods in Enzymology, 149:64-77; and Shafer-Korting M. et al., 1989, J. Am. Acad. Dermatol., 21:1271-1275). The preparation of liposomes and the variety of uses of liposomes in biological systems have been described (e.g., in U.S. Pat. No. 4,708,861 to M. C. Popescu et al., U.S. Pat. No. 4,224,179 to M. Schneider and U.S. Pat. No. 4,235,871 to D. P. Papahadjopoulos et al.). Accordingly, such liposomes can be formulated into any of the dermatological or cosmetic compositions as described herein.

In addition to the extracts from the flower of one or more species of the lotus flower constituents as active agents, as described herein, the physiologically acceptable and pharmaceutical compositions can contain suitable pharmaceutically acceptable carriers, diluents, or excipients comprising auxiliaries which facilitate processing of the active compounds into preparations which can be used pharmaceutically. Further details on techniques for formulation and administration are provided in the latest edition of Remington's Pharmaceutical Sciences (Mack Publishing Co.; Easton, Pa.). Pharmaceutical compositions containing the extracts from the flower of one or more species of the lotus flower ingredients of the present disclosure can be manufactured in a manner that is known in the art, e.g., by means of conventional mixing, dissolving, granulating, dragee-making, levigating, emulsifying, encapsulating, entrapping, or lyophilizing processes.

If applicable, the pharmaceutical composition can be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preparation can be a lyophilized powder.

A preferred aspect of the topical compositions of the present disclosure also includes at least one of the following: a surface smoother, a skin plumper, an optical diffuser, a sunscreen, an exfoliation promoter, or an antioxidant.

A surface smoother provides the functional benefits of enhancing skin smoothness and reducing the appearance of fine lines and coarse wrinkles. Examples include, without limitation, isopropyl myristate, petrolatum, isopropyl lanolate, silicones (e.g., methicone, dimethicone), or any mixtures thereof. The surface smoother is preferably present from about 0.1 wt % to about 50 wt % of the total weight of the composition.

A skin plumper serves as a collagen enhancer to the skin. An example of a suitable and preferred skin plumper is palmitoyl oligopeptide. Other nonlimiting examples of skin plumpers include collagen and/or glycosaminoglycan (GAG) enhancing agents. The skin plumper is preferably present from about 0.1 wt % to about 20 wt % of the total weight of the composition.

An optical diffuser is a particle that changes the surface optometrics of skin, resulting in a visual blurring and softening of, for example, lines and wrinkles. Examples of optical diffusers that can be used in the present disclosure include, but are not limited to, boron nitride, mica, nylon, polymethylmethacrylate (PMMA), polyurethane powder, sericite, silica, silicone powder, talc, Teflon, titanium dioxide, zinc oxide, or any mixtures thereof. The optical diffuser is preferably present from about 0.01 wt % to about 20 wt % of the total weight of the composition.

In another aspect, the present disclosure embraces a sunscreen that protects skin from damaging ultraviolet rays. Illustratively, the sunscreen provides both UVA and UVB protection by using either a single sunscreen or a combination of sunscreens. Among the sunscreens that can be employed in the present compositions are avobenzone, cinnamic acid derivatives (such as octylmethoxy cinnamate), octyl salicylate, oxybenzone, titanium dioxide, zinc oxide, or any mixture or combination thereof. Preferably, the sunscreen is present from about 1 wt % to about 30 wt % of the total weight of the composition. In particular, the addition of a sunscreen is preferred to prevent/reduce the photodegradation of the composition and/or ingredients therein while in the package and/or on the skin after application.

It will be appreciated that the compositions of the present disclosure containing sunscreen bring about additional improvements to the aesthetic appearance of skin, including at least one of the following: minimizes sunburning, minimizes tanning, reduces redness, and reduces future wrinkle development. It will be appreciated that when the topical composition is intended to be applied prior to retiring for the evening, the addition of a sunscreen agent may not be preferred.

The present compositions may also have one or more exfoliation promoters. Suitable examples of an exfoliation promoter that can be used in the present compositions include keratolytic agents, i.e., an active agent having desquamating, exfoliant, or scrubbing properties, or an active agent which can soften the horny layer of the skin; alpha (α) and/or beta (β) hydroxy acids; benzoyl peroxide; keto acids, such as pyruvic acid, 2-oxopropanoic acid, 2-oxobutanoic acid, and 2-oxopentanoic acid; oxa acids, as disclosed in U.S. Pat. Nos. 5,847,003 and 5,834,513, the disclosures of which are incorporated herein by reference; urea; retinoids, or any mixtures thereof. These anti-wrinkle or anti-fine line active agents can be formulated, for example, in amounts of from about 0.0001% to 5% by weight relative to the total weight of the composition.

More specifically, examples of hydroxy acids include, but are not limited to, α-hydroxy acids or β-hydroxy acids, either linear, branched, cyclic, saturated or unsaturated. The hydrogen atoms in the carbon-based backbone of these materials can be substituted with halogens, halogen-containing alkyl, acyl, acyloxy, alkoxycarbonyl, or alkoxy radicals having from 2 to 18 carbon atoms. Suitable hydroxy acids include, for example, glycolic acid, lactic acid, malic acid, tartaric acid, citric acid, 2-hydroxyalkanoic acid, mandelic acid, salicylic acid and alkyl derivatives thereof, including 5-n-octanoylsalicylic acid, 5-n-dodecanoylsalicylic acid, 5-n-decanoylsalicylic acid, 5-n-octylsalicylic acid, 5-n-heptyloxysalicylic acid, 4-n-heptyloxysalicylic acid and 2-hydroxy-3-methylbenzoic acid or alkoxy derivatives thereof, such as 2-hydroxy-3-methyoxybenzoic acid. Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof. Preferred exfoliation promoters are 3,6,9-trioxaundecanedioic acid, glycolic acid, lactic acid, or any mixtures thereof. Other acids, such as oxa acid (e.g., U.S. Pat. No. 6,069,169) and an oxa diacid (e.g., U.S. Pat. No. 5,932,229) can be included in the compositions of this disclosure.

When the present disclosure includes an exfoliation promoter, the composition typically includes about 0.5 wt % to 30 wt %, preferably about 1 wt % to about 15 wt %, more preferably about 2 wt % to about 10 wt %, and most preferably about 4 wt %, of the exfoliation promoter based on the total weight of the composition.

An antioxidant functions, among other things, to scavenge free radicals from skin to protect the skin from environmental agressors. Examples of antioxidants that can be used in the present compositions include compounds having phenolic hydroxy functions, such as ascorbic acid and its derivatives/esters; beta-carotene; catechins; curcumin; ferulic acid derivatives (e.g., ethyl ferulate or sodium ferulate); gallic acid derivatives (e.g., propyl gallate);

lycopene; reductic acid; rosmarinic acid; tannic acid; tetrahydrocurcumin; tocopherol and its derivatives; uric acid; or any mixtures thereof. Other suitable antioxidants are those having one or more thiol (—SH) functions, in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present disclosure can include an antioxidant preferably from about 0.001 wt % to about 10 wt %, and more preferably from about 0.001 wt % to about 5 wt %, of the total weight of the composition.

The compositions of this disclosure can also include one or more of the following ingredients, additives, or adjuvants: anesthetics, anti-allergenics, anti-fungals, antiseptics, chelating agents, colorants, dyestuffs, demulcents, emollients, emulsifiers, fragrances, fillers, humectants, lubricants, moisturizers, pH adjusters, pigment altering agents, preservatives, skin penetration enhancers, stabilizers, surfactants, thickeners, viscosity modifiers, hydrophilic or lipophilic gelling agents, vitamins, or any mixtures thereof. The amounts of these various ingredients, additives, adjuvants, or active agents are those that are conventionally used in the cosmetic or pharmaceutical fields, for example, they can constitute from about 0.01% to 20% of the total weight of the composition. In addition, the adjuvants, ingredients, additives, or active agents can be introduced into the fatty phase, into the liquid phase, and/or into lipid vesicles, depending on their nature.

The component(s) of the present disclosure are preferably contained in a cosmetically acceptable medium (i.e., vehicle, diluent or carrier). In an aspect embracing topical application, the compositions of this disclosure a medium that is compatible with human skin. The compositions can be formulated as aqueous, alcohol, or aqueous/alcohol-based solutions, ointments, lotions, gels, water-in-oil, oil-in-water, or water-oil-water triple emulsions having the appearance of a cream or gel, microemulsions, or aerosols. In addition, the compositions can be in the form of vesicular dispersions containing ionic and/or nonionic lipids, as described above. Dosage units suitable for such pharmaceutical compositions are formulated according to the conventional knowledge and techniques used in the art.

More particularly, the compositions for topical application can be in the form of a protective care composition for the skin, preferably for the face, the neck, the hands, the feet, or other areas of the body. Nonlimiting examples include day creams or lotions, night creams or lotions, sunscreen creams, lotions, or oils, body milks, makeup (a foundation), artificial tanning compositions and depilatories.

If the composition of the disclosure is an emulsion, the proportion of fatty substances comprised therein can range from about 5% to 80%, by weight, preferably from about 5% to 50% by weight, relative to the total weight of the composition. The fatty substances and emulsifiers used in the composition in emulsion form are one or more of those conventionally employed in the cosmetic or pharmaceutical field. Nonlimiting examples of fatty substances include mineral oils (e.g., petroleum jelly), plant oils and hydrogenated derivatives thereof, animal oils, synthetic oils (e.g., perhydrosqualene), silicone oils (e.g., polydimethylsiloxane) and fluoro oils. Other exemplary fatty substances include fatty alcohols (e.g., stearyl or cetyl alcohol), fatty acids (e.g., stearic acid) and waxes.

Emulsifiers are typically present in the compositions of the disclosure in an amount of about 0.3% to 30%, by weight and preferably from about 0.5% to 30% by weight relative to the total weight of the composition.

In one aspect, the present disclosure relates to the administration of an effective amount of at least one extracts from the flower of one or more species of the lotus flower constituent or composition comprised thereof to inhibit or block acetylcholine release by nerve cells at the NMJ to relax and/or decontract subcutaneous muscles, particularly facial expression muscles, thereby smoothing out or unwrinkling skin, wherein the inhibition of acetylcholine release by nerve cells at the NMJ corresponds to muscle, preferably facial expression muscle relaxation or decontraction.

In yet another aspect, the present disclosure encompasses a method of treating hyperhidrosis or perspiration, comprising applying to skin a composition containing extracts from the flower of one or more species of the lotus flower in a cosmetically and/or dermatologically acceptable medium, and in an amount effective to treat, reduce, prevent and/or ameliorate hyperhidrosis or perspiration. The application of the extracts from the flower of one or more species of the lotus flower containing composition is preferably topical.

Another aspect of the present disclosure relates to a method of improving the aesthetic appearance of skin and comprises applying to the skin, or introducing via a directed mode of delivery, a composition including one or more extracts from the flower of one or more species of the lotus flower constituents in an amount effective to improve the aesthetic appearance of the skin. According to this aspect in aesthetic appearance involves the treatment of at least one condition, such as signs of dermatological aging. Dermatological aging can include chronological aging, actinic aging, hormonal aging, or any combination thereof.

As will be appreciated by the practitioner, cosmetic treatments comprising compositions containing the extracts from the flower of one or more species of the lotus flower constituents of the disclosure can be carried out, for example, by topically applying the cosmetic composition as described herein according to the routine technique for administering such compositions. Routine and commonly practiced techniques encompass the application of creams, lotions, gels, sera, ointments, antiperspirants, or deodorants to the skin; spraying as a form of application is also envisioned.

In addition to the active ingredient, the disclosed cosmetic compositions may comprise any number of additional ingredients, such as, but not limited to: additional active ingredients (e.g., cosmetic, dermatological, and/or pharmaceutical), alcohols, allergy inhibitors, amino acids, anti-acne agents (e.g., salicylic acid), anti-aging agents, antiseptics, antifungal agents, s, analgesics, anti-hair loss agents, anti-wrinkle agents, antibacterial agents, anti-microbial agents, anti-oxidants, anti-inflammatory agents, burn healing agents, colorants (e.g., lakes, pigments, and the like), de-pigmentation agents, deodorants, dyes, emollient (e.g., glycerin, butylene glycol), excipients, fatty substances, fillers, film formers e.g., dimethicone acrylate copolymer, ethylhexyl acrylate copolymer), fragrances, free radical scavengers, glycerin, glycerin monostearate, glycerin distearate, hair growth agents, hair conditioners, hair softeners, hair moisturizers, herbal extracts, humectants (e.g., hyaluronic acid, orotic acid, lipoprotein), insect repelants, medication, moisturizers, non-active carrier oils (e.g., triglycerides, silicone oils, mineral oils), oils, peptides, polypeptides, proteins, perfumes, pigments, preservatives, plasticizers, reflectants, sebum absorbers, skin lightening agents, sunscreens, surfactants, tanning agents, thickening agents hydroxyethylcellulose, xanthan gum, carbomer), Vaseline, vasoconstrictors, vasodilators, vitamins (e.g., Vitamin A, Vitamin E), water, waxes, and/or combinations thereof.

In addition to the active ingredient, the disclosed cosmetic compositions may comprise any number of additional ingredients, such as, but not limited to: additional active ingredients (e.g., cosmetic, dermatological, and/or pharmaceutical), alcohols, allergy inhibitors, amino acids, anti-acne agents (e.g., salicylic acid), anti-aging agents, antiseptics, antifungal agents, s, analgesics, anti-hair loss agents, anti-wrinkle agents, antibacterial agents, anti-microbial agents, anti-oxidants, anti-inflammatory agents, burn healing agents, colorants (e.g., lakes, pigments, and the like), de-pigmentation agents, deodorants, dyes, emollient (e.g., glycerin, butylene glycol), excipients, fatty substances, fillers, film formers e.g., dimethicone acrylate copolymer, ethylhexyl acrylate copolymer), fragrances, free radical scavengers, glycerin, glycerin monostearate, glycerin distearate, hair growth agents, hair conditioners, hair softeners, hair moisturizers, herbal extracts, humectants (e.g., hyaluronic acid, orotic acid, lipoprotein), insect repelants, medication, moisturizers, non-active carrier oils (e.g., triglycerides, silicone oils, mineral oils), oils, peptides, polypeptides, proteins, perfumes, pigments, preservatives, plasticizers, reflectants, sebum absorbers, skin lightening agents, sunscreens, surfactants, tanning agents, thickening agents hydroxyethylcellulose, xanthan gum, carbomer), Vaseline, vasoconstrictors, vasodilators, vitamins (e.g., Vitamin A, Vitamin E), water, waxes, and/or combinations thereof.

Sunscreens components can include zinc oxide, titanium dioxide, octinoxate, ensulizole, ethylhexyl triazone, octyl salicylate, butyl methoxydibenzoylmethane, bisethylhexyloxyphenol methoxyphenyl triazine, isoamyl-p-methoxycinnamate, diethylhexyl butamido triazone, or a mixture thereof. In some aspects, the sunscreen components can be free of octocrylene. Additionally, according to some aspects, the sunscreen formulations described herein are eco-compatible compositions. For example, the sunscreen formulations are compatible with organism life and not harmful to marine ecosystems (in particular for tropical environments with coral reefs). Specifically, the sunscreen formulations are free of components that inhibit cell growth or cell replication in a marine environment. In some examples, this can be accomplished by the sunscreen formulations being free of bis ethylhexyloxyphenol methoxyphenyl triazine (BEMT), cinnamates, benzophenones, camphor derivatives, titanium oxide, melaleuca essential oil, ylang ylang flower extracts, Bambousa Arundinacea extracts, Karitè, animal-derived fats, argan oil, ocopheryl acetate, tocopherol, retinol, polyphenolic compounds extracted from green tea or mixtures thereof.

The composition of the present disclosure may also include other cosmetic ingredients such as, but not limited to, humectants emollients, moisturizers, anti-wrinkle ingredients, concealers, matte finishing agents, pigments, colorants, proteins, anti-oxidants, bronzers, chelating agents, emulsifiers, ultraviolet (UV) absorbing agents, oil absorbing agents, anti-foam agents, anti-tack agents, thickeners, fragrances, preservatives, anti-microbials, fungistats, neutralizing agents, vitamins, plasticizers, cohesion agents, basifying and acidifying agents, fillers, solvents, and mixtures thereof.

The compositions may contain additional ingredients such as alkalinizing agents, emulsifying agents, emollients, plasticizers, preservatives, humectants, moisturizing agents, solvents, and tonicity agents or active ingredients suitable to provide anti-aging benefits. Examples of preferred additional ingredients include glycerin.

Additional ingredients may optionally be added to the disclosed compositions as detailed below.

Colorants or pigments: The compositions may comprise one or more cosmetic powders, for example, calcium aluminum borosilicate, PMMA, polyethylene, polystyrene, methyl methacrylate crosspolymer, nylon-12, ethylene/acrylic acid copolymer, boron nitride, Teflon, silica, or the like. Typically the compositions will include colorants or pigments to impart a desired color or effect, examples are inorganic pigments, organic pigments, and/or lakes. Exemplary inorganic pigments include, but are not limited to, metal oxides and metal hydroxides such as magnesium oxide, magnesium hydroxide, calcium oxide, calcium hydroxides, aluminum oxide, aluminum hydroxide, iron oxides (α-Fe₂O₃, γ-Fe₂O₃, Fe₃O₄, FeO), red iron oxide, yellow iron oxide, black iron oxide, iron hydroxides, titanium dioxide, titanium lower oxides, zirconium oxides, chromium oxides, chromium hydroxides, manganese oxides, cobalt oxides, cerium oxides, nickel oxides and zinc oxides and composite oxides and composite hydroxides such as iron titanate, cobalt titanate and cobalt aluminate. Non-metal oxides such as alumina and silica, ultramarine blue (i.e., sodium aluminum silicate containing sulfur), Prussian blue, manganese violet, bismuth oxychloride, talc, mica, sericite, magnesium carbonate, calcium carbonate, magnesium silicate, aluminum magnesium silicate, silica, titanated mica, iron oxide titanated mica, bismuth oxychloride, and the like, are also contemplated to be suitable inorganic pigments. Organic pigments can include, but are not limited to, at least one of carbon black, carmine, phthalocyanine blue and green pigment, diarylide yellow and orange pigments, and azo-type red and yellow pigments such as toluidine red, litho red, naphthol red and brown pigments, and combinations thereof.

Lakes generally refer to a colorant prepared from a water-soluble organic dye, (e.g., D&C or FD&C) which has been precipitated onto an insoluble reactive or adsorptive substratum or diluent. The term “D&C” as used herein means drug and cosmetic colorants that are approved for use in drugs and cosmetics by the FDA. The term “FD&C” as used herein means food, drug, and cosmetic colorants which are approved for use in foods, drugs, and cosmetics by the FDA. Certified D&C and FD&C colorants are listed in 21 C.F.R. § 74.101 et seq. and include the FD&C colors Blue 1, Blue 2, Green 3, Orange B, Citrus Red 2, Red 3, Red 4, Red 40, Yellow 5, Yellow 6, Blue 1, Blue 2, Orange B, Citrus Red 2, and the D&C colors Blue 4, Blue 9, Green 5, Green 6, Green 8, Orange 4, Orange 5, Orange 10, Orange 11, Red 6, Red 7, Red 17, Red 21, Red 22, Red 27, Red 28, Red 30, Red 31, Red 33, Red 34, Red 36, Red 39, Violet 2, Yellow 7, Yellow 8, Yellow 10, Yellow 11, Blue 4, Blue 6, Green 5, Green 6, Green 8, Orange 4, Orange 5, Orange 10, Orange 11, and so on. Substrates suitable for forming lakes include, without limitation, mica, bismuth oxychloride, sericite, alumina, aluminum, copper, bronze, silver, calcium, zirconium, barium, and strontium, titanated mica, filmed silica, spherical silica, polymethylmethacrylate (PMMA), micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorilionite, microcrystalline cellulose, rice starch, silica, talc, mica, titanium dioxide, zinc laurate, zinc myristate, zinc rosinate, alumina, ahapulgite, calcium carbonate, calcium silicate, dextran, nylon, silica silylate, silk powder, sericite, soy flour, tin oxide, titanium hydroxide, trimagnesium phosphate, walnut shell powder, and mixtures thereof. Suitable lakes include, without limitation, those of red dyes from the monoazo, disazo, Norm, xanthene, or indigoid families, such as Red 4, 6, 7, 17, 21, 22, 27, 28, 30, 31, 33, 34, 36, and Red 40; lakes of yellow pyrazole, monoazo, fluoran, xanthene, quinoline, dyes or salt thereof, such as Yellow 5, 6, 7, 8, 10, and 11; lakes of violet dyes including those from the anthroquinone family, such as Violet 2, as well as lakes of orange dyes, including Orange 4, 5, 10, 11, and the like. Suitable lakes of D&C and FD&C dyes are defined in 21 C.F.R. § 82.51.

The coloring agents may be optionally surface treated, for example, to make the particles more hydrophobic or more dispersible in a vehicle. The surface of the particles may, for example, be covalently or ionically bound to an organic molecule or silicon-based molecule or may be adsorbed thereto, or the particle may be physically coated with a layer of material. The surface treatment compound may be attached to the particle through any suitable coupling agent, linker group, or functional group (e.g., silane, ester, ether, etc). The compound may comprise a hydrophobic portion which may be selected from, for example, alkyl, aryl, ailyl, vinyl, alkyl-aryl, aryl-alkyl, organosilicone, di-organosilicone, dimethicones, methicones, polyurethanes, silicone-polyurethanes, and fluoro-or perfluoro-derivatives thereof. Other hydrophobic modifiers include lauroyl lysine, isopropyl Titanium Triisostearate (ITT), ITT and Dimethicone (ITT/Dimethicone) ITT and Amino Acid, cross-polymers,

ITT/Triethoxycaprytylsilarte Crosspolymer, waxes (e.g., carnauba), fatty acids (e.g., stearates), HDI/Trimethylol Hexylactone Crosspolymer, PEG-8 Methyl. Ether Triethoxysilane, aloe, jojoba ester, lecithin, perfluoroalcohol phosphate, and Magnesium Myristate (MM), to name a few.

An optional pigment component includes and alkyl silane surface-treated colorant consisting essentially of or comprising an alumina substrate (e.g., platelet shaped) and a pigment, dye, or lake bonded to the alumina substrate by an alkyl silane surface treatment, Typically, the alkyl slime will be octylsilane, and may be formed by treatment with triethoxy caprylylsilane. Non-limiting examples of such colorants include, but are not limited to, Alumina/Titanium Dioxide/Triethoxycaprylylsilane 1% (COVALUMINE Atlas White AS), Alumina/D&C Red Aluminum Lake CTD/Triethoxycaprylylsilane 1% (COVALUMINE Red

Rose AS), Alumina/D&C Red Aluminum Lake CTD/Triethoxycaprylylsilane 1% (COVALUMINE Sonoma Red AS), Alumina/Black Iron Oxide CTD/Triethoxycaprylylsilane 1% (COVALUMINE Sonoma Black AS), Alumina/D&C Red #6 Aluminum Lake CTD/Triethoxycaprylylsilane 1% (COVALUMINE Fire Red AS), Alumina/Yellow Iron Oxide CTD/Triethoxycaprylylsilane 1% (COVALUMINE Sonoma Yellow AS), Alumina/D&C Blue #1 Aluminum Lake CTD/Triethoxycaprylylsilane 1% (COVALUMINE Astral Blue AS), Alumina/Carmine CTD/Triethoxycaprylylsilane 1% (COVALUMINE Campari AS), Alumina/Yellow #5 CTD/Triethoxycaprylylsilane 1% (COVALUMINE Sunburst AS), Alumina/Triethoxycaprylylsilane 1%, and combinations thereof, each of which is available from SENSIENT Cosmetic Technologies LCW.

Interference or pearl pigments may also be included. These are typically comprised of micas layered with about 50 to 300 nm films of TiO₂, Fe₂O₃, or Cr₂O₃ or the like. These include white nacreous materials, such as mica covered with titanium oxide or covered with bismuth oxychloride; and colored nacreous materials, such as titanium mica with iron oxides, titanium mica with ferric blue or chromium oxide, titanium mica with an organic pigment of the aforementioned type. If these materials are used, it is preferred that these materials are used collectively in an amount of less than 1.0 wt %. Preferably, the pearlescent component has a bismuth oxychloride based pearlescent ingredient or reflectance pearls. Bismuth oxychloride matches the skin's natural pearlescence more than compounds such as titanium oxide, which provide for a more artificial look. Bismuth oxychloride better mimics the skin's natural reflectance. However, other pearlescent ingredients may be used. A preferred pearlescent component is called CHROMA-LITE, which is a combination of colored pigment bonded to BI-LITE 20 (bismuth oxychloride and mica) using calcium stearate. The CHROMA-LITE component is available in various shades/colors from Englehard Corporation (Iselin, N.J.).

Cosmetically acceptable vehicles: The disclosed compositions will typically comprise a cosmetically acceptable vehicle. “Cosmetically acceptable” as used herein means that it is safe for contact with a human integument. The vehicle may comprise a liquid, comprising a single phase, a dual-phase system, or an emulsion. Emulsions include oil-in-water, silicone-in-water, water-in-oil, water-in-silicone, and the like. When formulated as an emulsion, an emulsifier is typically included. Special mention may be made of volatile silicones (e.g., cyclopentasiloxane), hydrocarbons, ester oils, lower alcohols (e.g., ethanol, isopropyl alcohol, etc.), and water.

Emollients: Emollients may be used in the disclosure, and include, but are not limited to, esters oils, which will typically be the etherification product of an acid of the form R₄(COOH)₁₋₂ with an alcohol of the form R₅(OH)₁₋₃ where R₄ and R₅ are each independently linear, branched, or cyclic hydrocarbon groups, optionally containing unsaturated bonds, and having from 1 to 30 carbon atoms, preferably from 2 to 30 carbon atoms, and more preferably, from 3 to 30 carbon atoms, optionally substituted with one or more functionalities including hydroxyl, oxa, oxo, and the like. Preferably, at least one of R₄ and R₅ comprises at least one fatty chain. The esters defined above will include, without limitation, the esters of mono-acids with mono-alcohols, mono-acids with diols and triols, di-acids with mono-alcohols, and tri-acids with mono-alcohols. Other emollients include dimethicone. Humectants, such as glycerin and other C1-10 polyols or diols may also be included.

Absorbent Powders: Absorbent powders capable of absorbing oils for increased comfort can be added. Examples of suitable absorbent powders can include silica, mica, elastomers, kaolin, starch, or mixtures thereof.

Fillers: Fillers can also optionally be added, in an amount from about 1% to about 20%, preferably from about 1% to about 10% by weight of the final composition. Examples of fillers include, but are not limited to, silica, PMMA, nylon, alumina, barium sulfate, or any other filler typically used in such compositions.

Film formers: Polymeric film formers include cellulosics, polyolefins, polyvinyls, polacrylates, polyurethanes, silicones, silicone acrylates, polyamides, polyesters, fluoropolymers, polyethers, polyacetates, polycarbonates, polyimides, rubbers, epoxies, formaldehyde resins, and homopolymers and copolymers of any of the foregoing. In particular, When present, the film former can be in a range of from about 2 wt % to about 20 wt % of the active component, about 7 wt % to about 13 wt % of the active component, less than, equal to, or greater than about 2 wt %, 3, 4, 5, 6, 7, 8, 9, 10, 11, 12, 13, 14, 15, 16, 17, 18, 19, or about 20 wt %. As described herein above, the film former can be used to help produce a homogenous mixture of the extract solution as well as help to evenly distribute the composition about a user's skin. According to various aspects, the film former can include Acrylates Copolymer, Acrylates Copolymer (and) Acrylates/Polytrimethylsiloxymethacrylate Copolymer, ACRYLATES/OCTYLACRYLAMIDE COPOLYMER, Acrylates/C1-2Succinates/Hydroxyacrylates Copolymer, Acrylates/C12-22 Alkyl Methacrylate Copolymer, Acrylates/Dimethicone Copolymer, Acrylates/Ethylhexyl Acrylate Copolymer, Acrylates/Hydroxyesters Acrylates Copolymer, Acrylates/Lauryl Acrylate/Stearyl Acrylate/Ethylamine Oxide Methacrylate Copolymer, Acrylates/Octylacrylamide Copolymer Acrylates/Polytrimethylsiloxymethacrylate Copolymer, Acrylates/Polytrimethylsiloxymethacrylate Copolymer (and) Laureth-1 Phosphate, Acrylates/t-Butylacrylamide Copolymer, Adipic Acid/Diglycol Crosspolymer, Adipic Acid/Neopentyl Glycol/Trimellitic Anhydride Copolymer, Algin Aluminum Starch OctenylsuccinateAMP-Acrylates Copolymer, Behenyl Methacrylate/t-Butyl Methacrylate Copolymer, Brassica Campestris/Aleurites Fordi Oil Copolymer, Butyl Acrylate/Hydroxypropyl Dimethicone Acrylate Copolymer, Butyl Ester of PVM/MA Copolymer, C24-28 Alkyldimethylsiloxy Trimethylsiloxysilicate, Capryloyl Glycerin/Sebacic Acid Copolymer, Diisostearoyl Polyglyceryl-3 Dimer Dilinoleate, Ethyl Ester of PVM/MA Copolymer, Isobutylene/Ethylmaleimide/Hydroxyethylmaleimide Copolymer, Isopropyl Ester of PVM/MA Copolymer, Methacryloyl Ethyl Betaine/Acrylates Copolymer, Octylacrylamide/Acrylates/Butylaminoethyl Methacrylate Copolymer, Phenylpropyldimethylsiloxysilicate, Polyamide-3, Polyamide-8, Polyester-10, Polyester-7, Polyethylene, Polymethylsilsesquioxane, Polyurethane-1, Polyurethane-14 (and) AMP-Acrylates Copolymer, Polyurethane-18, Polyurethane-34, Polyurethane-35, Polyurethane-48, Polyurethane-64, Polyurethane-93, Polyvinyl Stearyl Ether, PPG-17/IPDI/DMPA Copolymer, PPG-26/Dimer Dilinoleate Copolymer, Pullulan, PVP, PVP CROSSPOLYMER, Sodium Polyacrylate, Sodium Polystyrene Sulfonate, Styrene/Acrylates Copolymer, Styrene/VP Copolymer, Triacontanyl PVP, Trifluoropropyldimethylsiloxy/Trimethylsiloxy Silsesquioxane, Trimethylpentanediol/Adipic Acid/Glycerin Crosspolymer, Trimethylsiloxysilicate, VA/Butyl, Maleate/Isobornyl Acrylate Copolymer, VA/Crotonates Copolymer, VA/Crotonates/Vinyl Neodecanoate Copolymer, Vinyl Caprolactam/VP/Dimethylaminoethyl Methacrylate Copolymer, VP/HEXADECENE COPOLYMER, VP/Acrylates/Lauryl Methacrylate Copolymer, VP/Dimethylaminoethylmethacrylate Copolymer, VP/DMAPA Acrylates Copolymer, VP/Eicosene Copolymer, VP/Hexadecene Copolymer, VP/Methacrylamide/Vinyl Imidazole Copolymer, VP/VA Copolymer, VP/Vinyl Caprolactam/DMAPA Acrylates Copolymer, copolymers thereof, or mixtures thereof. In specific aspects, the film former can include sodium polyacrylate.

Oils: Suitable non-limiting examples of oils for the oil phase (for example, in an emulsion) include natural and synthetic oils, including animal, vegetable, and petroleum oils; fatty acid triglycerides; fatty acid esters such as octyl palmitate, isopropyl myristate and isopropyl palmitate; ethers such as dicapryl ether; fatty alcohols such as cetyl alcohol, stearyl alcohol and behenyl alcohol; sterols; hydrocarbons such as isooctane, isododecane, isohexadecane, decane, dodecane, tetradecane, tridecane, C₈₋₂₀ isoparaffins, mineral oil, petrolatum, isoeicosane and polyisobutene; C₁₀₋₃₀ cholesterol/lanosterol esters; lanolin; and the like. Representative hydrocarbons include paraffinic hydrocarbons available from Exxon under the ISOPARS trademark, and from the Permethyl Corporation. In addition, C₈₋₂₀ paraffinic hydrocarbons such as C₁₂ isoparaffin (isododecane) manufactured by the Permethyl Corporation having the tradename PERMETHYL 99A™. are also contemplated to be suitable. Various commercially available C₁₆ isoparaffins, such as isohexadecane (having the tradename PERMETHYL.R™) are also suitable. Silicone oils such as dimethicones, cyclic silicones, and polysiloxanes may also be included in the oil phase. In one aspect, silicone oils are present in an amount less than about 5% by weight of the oil phase.

Solvents: The cosmetically acceptable vehicle may comprise a volatile solvent. Typically, a volatile solvent may have a vapor pressure of above about 0.01 mmHg at 20. degree. C. Volatile solvents may include volatile C₅₋₁₂ hydrocarbons (e.g., isododecane), aromatic hydrocarbons (e.g., xylenes, toluene, etc.), ketones acetone, methylethyl ketone, etc.), ethers (e.g., diethyl ether, methylethyl ether, etc.), perfluorohydrocarbons, hydrofluoroethers,

Freons, esters of acetic acid (e.g., ethylacetate, butylacetate, etc.) and the like. Preferred volatile solvents will be cosmetically acceptable. The solvent can be an organic solvent. Where present, the solvent is in a range of from about 10 wt % to about 50 wt % of the active component, about 30 wt % to about 40 wt % of the active component, less than, equal to, or greater than about 30 wt %, 31, 32, 33, 34, 35, 36, 37, 38, 39, 40, 41, 42, 43, 44, 45, 46, 47, 48, 49, or about 50 wt % of the composition. Examples of organic solvents can include an isododecane component, an alkyl benzoate component, and a neopentyl glycol diheptanoate component. Isododecane (an example of which is represented by CAS 31807-55-3) is generally understood to be a clear, colorless and odorless, volatile liquid, which makes it suitable for use in color cosmetics like mascara, eyeliner, lip products, or any product where improved wear properties and no residues are wanted. It does not leave an oily residue. Isododecane is a volatile, lipophilic component for deodorant sprays and hair care applications. It is a hydrocarbon ingredient used as a solvent. The alkyl benzoate component can be C12-15 alkyl benzoate (an example of which is represented by CAS 68411-27-8), which is generally understood to be a low-molecular weight ester of benzoic acid and C12-15 alcohols. C12-15 alkyl benzoate is a clear liquid that is practically odorless. In some examples, the alkyl benzoate component includes a C12 alkyl benzoate, a C13 alkyl benzoate, a C14 alkyl benzoate, a C15 alkyl benzoate, or a mixture thereof. Neopentyl glycol diheptanoate (an example of which is represented by CAS 68855-18-5) is generally understood to be a mixture of texture-enhancing ingredient neopentyl glycol and grape-derived fatty acid heptanoic acid.

According to various aspects of the present disclosure, the composition is substantially free of cyclopentasiloxane. For example, the composition can include less than about 10 wt % cyclopentasiloxane, less than about 9 wt %, less than about 8 wt %, less than about 7 wt %, less than about 6 wt %, less than about 5 wt %, less than about 4 wt %, less than about 3 wt %, less than about 2 wt %, less than about 1 wt %, less than about 0.5 wt %, less than about 0.1 wt %, less than about 0.01 wt %, or less than about 0.001 wt %. in some aspects, the composition is completely free (e.g., includes 0 wt %) of cyclopentasiloxane. The disclosed isododecane component, alkyl benzoate component, and neopentyl glycol diheptanoate component, together, function to effectively replace cyclopentasiloxane in an while having similar, equal, or superior performance. Replacing cyclopentasiloxane can be desirable because, despite its performance in compositions, regulatory schemes seek to limit cyclopentasiloxane's use in cosmetic products. Therefore, it is important to find an effective substitute for cyclopentasiloxane. Suitable substitutes can include an oleochemical component such as dicaprylyl carbonate, dicaprylyl ether, or a mixture thereof.

The surface of all or a portion of the individual pigments of the pigment components can be at least partially coated. Examples of suitable coatings include a lauroyl lysine coating, a silane coating, a methicone coating, a dimethicone coating, a stearoyl glutamic acid, or a combination thereof. As generally understood, a lauroyl lysine is an amino acid derivative, derived from natural product lysine a protein and lauric acid another natural coconut fatty acid. lauroyl lysine treatment creates hydrophobic system with enhanced dispersion, increased wear properties and make up with a wet feel on the skin. Pigment surfaces are smooth, flat and less fluffy than with silane treatment, thus easier to press. Lauroyl lysine coatings are lubricious, giving the substrate a smooth, moist feel. As generally understood, a silane coating is a deposition of coatings through the reaction of silanols with the pigment. By hydrolysis of “Si—OR” groups to silanol (Si—OH), alkyl silane can self-associate or hydrogen bond to the pigment surface, so stable bonds are formed between the pigment and the treating compound following curing. Silicone treated pigments disperse well in cyclomethicones. They have very low surface tension, so they have excellent hydrophobicity and better lipophilicity due to which they easily disperse in mineral oils, esters and silicone fluids. Pigments treated with alkyl silane are more hydrophobic than methicone treated pigments, wet better in commonly utilized cosmetic oils and have lower oil absorption. In hydrous compact formulations, the excellent wetting imparted by alkyl silane treatment allows incorporation of high pigment loads to achieve a “powdery” sensation upon application to the skin while maintaining a low melt viscosity for hot filling. As generally understood, a methicone coating is a polymethylhydrogen siloxane, which absorb traces of water from pigment surface and converts Si—H bond to Si—OH with liberation of small amount of hydrogen gas and upon curing form covalent Si-0 pigment chemical bonds. The coating formed is highly hydrophobic and not solubilized by common solvents. The pigments wet well in oils particularly silicone oils. Skin feel is somewhat dry with enhanced slip and spreadability. As generally understood, a dimethicone coating (also called polymethylsiloxane) is a coating bonded to a pigment or filler surface via the mechanism of hydrolysis, condensation and curing to a Si-O pigment linkage. Surface treated with dimethicone are quite hydrophobic and have good slip and more lubricious feel. It is useful in oil based systems, which may be used for anhydrous products.

On individual pigments, the coating can substantially coat 100% of the total surface area of the individual pigment. In some further aspects, the coating can coat about 5% to about 95% of the total surface area of the individual pigment about 10% of the total surface area to about 90% of the total surface area, about 15% of the total surface area to about % of the total surface area, about 20% of the total surface area to about 80% of the total surface area, about 25% of the total surface area to about 75% of the total surface area, about 30% of the total surface area to about 70% of the total surface area, about 35% of the total surface area to about 65% of the total surface area, about 40% of the total surface area to about 60% of the total surface area, about 45% of the total surface area to about 50% of the total surface area, less than, equal to, or greater than about, 5% of the total surface area, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or about 95% of the total surface area. In some aspects, at least a portion of the total number of pigments present may be substantially free of any coating. For example, when less than 100% of the total number of pigments include a coating about 5% to about 95% of the total number of pigments can include a coating, about 10% to about 90% of the total number of pigments can include a coating, about 15% to about % of the total number of pigments can include a coating, about 20% to about 80% of the total number of pigments can include a coating, about 25% to about 75% of the total number of pigments can include a coating, about 30% to about 70% of the total number of pigments can include a coating, about 35% to about 65% of the total number of pigments can include a coating, about 40% to about 60% of the total number of pigments can include a coating, about 45% to about 50% of the total number of pigments can include a coating, less than, equal to, or greater than about, 5% of the total number of pigments can include a coating, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or about 95%. Each pigment can be coated with the same material or a different portions of the pigments can be at least partially coated with different coatings. An example of a suitable coating is stearoyl glutamic acid.

In some examples in which the coating is stearoyl glutamic acid, the composition includes Triglycerol diisosterarate, glyceryl stearate, or a combination thereof.

Thickeners: Suspending and thickening agents typically include silica gels, gums, clays, fumed silica, fatty acid soaps, and various hydrocarbon gels, and other ingredients that when incorporated into the formulation remain on the surface of keratinous tissues as disclosed in the International Cosmetic Dictionary and Handbook (12th Ed.), which is hereby incorporated by reference. Viscosifying agents such as gellants may also be used. Examples include bentone, triglycerides, aluminum stearate, C18-C36 acid glycol esters, glyceryl tribehenate, glycerol monostearate, alginates, carbomers, celluloses, gums; carageenans, starches or silicates.

Waxes: Waxes which may be used in the disclosure include, but are not limited to, linear polyethylene, microcrystalline petroleum wax, carnauba wax, lignite wax, ouricouri wax, rice bran wax, castor wax, mortar wax, stearone, acrawax, bayberry wax, castor wax,

Japan wax, ozokerite, beeswax, candelilla wax, petrolatum, ceresin wax, cocoa butter, illipe butter, esparto wax, shellac wax, ethylene glycol diesters or triesters of C18-C36 fatty acids, cetyl palmitate, paraffin wax, hard tallow, lanolin, lanolin alcohol, cetyl alcohol, glyceryl monostearate, sugarcane wax, jojoba wax, stearyl alcohol, silicone waxes, and combinations thereof.

In some aspects of the present disclosure, the composition can include a preservative. Where present, the preservative can be a range of from about 0.0001 wt % to about 0.5 wt % of the composition, about 0.0003 wt % to about 0.1 wt %, less than, equal to, or greater than about 0.0001 wt %, 0.0003, 0.0005, 0.001, 0.005, 0.01, 0.05, 0.1, or about 0.5.

When included, the preservative is included in an amount sufficient to prevent spoilage or prevent growth of inadvertently added microorganisms for a specific period of time, but not sufficient enough to contribute to the odor neutralizing performance of the composition. In other words, the preservative is not being used as the antimicrobial compound to kill microorganisms on the surface onto which the composition is deposited in order to eliminate odors produced by microorganisms. Instead, it is being used to prevent spoilage of the composition in order to increase shelf-life.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis {N′-[1-(hydroxymethyl)-2,5-dioxo-4-imidazolidinyl] urea}, commonly known as imidazolidinyl urea, available, e.g., under the trade name Abiol® from 3V-Sigma, Unicide U-13® from Induchem, Germall 115® from Sutton Laboratories, Inc.; polymethoxy bicyclic oxazolidine, available under the trade name Nuosept® C from Hüls America; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation.

The composition can further include an inactive component. The inactive component can be in a range of from about 50 wt % to about 90 wt % of the composition, about 75 wt % to about 85 wt % of the composition, less than, equal to, or greater than about 50 wt %, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or about 90 wt %. when the inactive component is present, the active component is in a range of from about 10 wt % to about 50 wt % of the composition, about 15 wt % to about 25 wt % of the composition, less than, equal to, or greater than about 10 wt %, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 wt % of the composition.

In one aspect, the composition is a pretreatment composition which consists of a nonpigmented formulation containing an active component for application to the integument for the purpose of sebum reduction prior to application of a colored cosmetic composition. Any portion of the aforementioned constituents can be combined in any desirable manner to produce many different types of cosmetic compositions. The cosmetic compositions can be applied to the epithelial layer of a user's skin. For example the cosmetic composition can be a pretreatment formulation, make-up formulation, a foundation, a sunscreen, or any other cosmetic applied to a user's epithelial tissue. Additionally, any portion of the aforementioned constituents can be combined to create a cosmetic formulation that can be applied to a keratinous tissue of a user. For example, the cosmetic formulation can be a mascara, hair dye, nail polish, lip gloss, lipstick or other pigmented/colored cosmetic formulation intended to be a long-wear composition.

### EXAMPLES

The following examples are provided as prophetic examples of potential use and results from testing of formulas that utilize a sebum/sweat reducing agent such as NELUPURE (INCI: PROPANEDIOL (AND) GLYCERIN (AND) NYMPHAEA CAERULEA FLOWER EXTRACT (AND) NELUMBO NUCIFERA FLOWER EXTRACT) as compared to control formulas that do not utilize such as sebum/sweat reducing agent. Various aspects of the present disclosure can be better understood by reference to the following Examples which are offered by way of illustration. The present disclosure is not limited to the Examples given herein.

### Example 1: Consumer Comparison of Long Wear Foundation Compositions

In a consumer test survey, consumers will be asked to compare a foundation composition comprising the NELUPURE (INCI: PROPANEDIOL (AND) GLYCERIN (AND) NYMPHAEA CAERULEA FLOWER EXTRACT (AND) NELUMBO NUCIFERA FLOWER EXTRACT) extract solution to a commercialized transfer resistant, foundation composition (Standard) which does not contain the NELUPURE (INCI: PROPANEDIOL (AND) GLYCERIN (AND) NYMPHAEA CAERULEA FLOWER EXTRACT (AND) NELUMBO NUCIFERA FLOWER EXTRACT) extract solution.

Each of the consumer testers will apply a layer of each foundation composition onto different clean (i.e., without wearing any face products) halves of their faces (e.g., left side vs. right side) in a double blind test. The consumers will be asked to compare the feel, comfort, appearance/coverage, and ease of removal of each at different times: initially after application, at 8 hours, and up to 9.9 hours after application and answer questions in a questionnaire comparing the two foundation compositions. Data will be tabulated using the Compusense5 program and analyzed using standard statistical processing programs.

The comments from the consumer tests described above are expected to indicate that overall, the foundation composition comprising NELUPURE (INCI: PROPANEDIOL (AND) GLYCERIN (AND) NYMPHAEA CAERULEA FLOWER EXTRACT (AND) NELUMBO NUCIFERA FLOWER EXTRACT) extract solution will provide a significantly more natural look after 8 hours of wear compared to the other Standard foundation. The consumers are expected to find that the feel of the novel foundation is significantly more lightweight and comfortable at 8 hours after application when compared to the Standard. The consumers are expected to think that the disclosed foundation was breathable and comfortable. As for the appearance or coverage, the consumers are expected to agree that the disclosed foundation evened out skin tone, reduced the appearance of lines and wrinkles, reduced redness, and reduced the appearance of age or sun spots. Overall, it is expected that the disclosed foundation provided a natural and flawless appearance.

The technical evaluator who oversees the consumer testing is expected to determine that the novel foundation was more visible and provided more coverage at hours after application than the Standard foundation. On average, the novel foundation is expected to last at least 8 hours. The technical evaluator is expected to deem the novel foundation composition to be “long wearing.”

### Example 2: Consumer Testing of Long Wear Foundation Composition

In a consumer test survey, consumer testers will be asked to evaluate the inventive foundation composition comprising the NELUPURE (INCI: PROPANEDIOL (AND) GLYCERIN (AND) NYMPHAEA CAERULEA FLOWER EXTRACT (AND) NELUMBO NUCIFERA FLOWER EXTRACT) extract solution. Each of the consumer testers will apply a layer of the foundation composition onto their clean faces (i.e., without wearing any face products). The consumers will be asked to evaluate the feel, comfort, application, appearance, and coverage at different times: initially at application and after 8 hours of application and answer questions. The questionnaire will be comprised of a series of questions about the aesthetics and performance of the foundation based on a seven point attribute scale. An Expert Evaluator will also rate the appearance and wear properties at the initial time point and after 8 hours of wear compared to a Standard.

Overall, results along the following general description are expected: the consumers will like the composition initially and after 8 hours of wear. They will find that the foundation was easy to apply and blend, providing even coverage and a smooth feel upon application. They will find that the composition was initially lightweight; evened out skin tone; reduced: the appearance of fine lines and wrinkles, appearance of redness, dark under eye circles, age/sun spots, and shine; improved the appearance of skin texture; and provided a flawless and natural look. While after 8 hours of wear, the foundation is expected to be found to be breathable and comfortable. The finish, coverage, and color were expected to be maintained after 8 hours of wear. The foundation composition is expected to still control shine and oil breakthrough, while appearing freshly applied. After 8 hours the consumers are expected to find that the foundation did not settle in lines, wrinkles or pores, nor look cakey or masky. Even though it is expected that the foundation did not transfer after 8 hours, it is expected that the significant majority of the consumers will find that the foundation composition is easy to remove.

### Example 3: Inhibition of Acetylcholine Release at the Neuromuscular Junction

The aim of the study is to assess the effect of Nelupure (INCI: Propanediol (and) Glycerin (and) Nymphaea caerulea Flower Extract (and) Nelumbo Nucifera Flower Extract) at 0.2%. on the inhibition of Acetylcholine release. The study will be performed with a specific neuromuscular co-culture model. In this model, human fibroblasts will be reprogrammed into pluripotent cells and then differentiated into Motor Neurons. These Motor Neurons will be cocultured with human muscle cells, creating a functional neuro-muscular junctions. After incubation with Nelupure (INCI: Propanediol (and) Glycerin (and) Nymphaea caerulea Flower Extract (and) Nelumbo Nucifera Flower Extract) @ 0.2%., cultures will be activated by glutamate, to induce Acethylcholine release by motor neurons. After activation, Acetylcholine release will be quantified in the cell culture medium by appropriate ELISA test. Under these experimental conditions, it is expected to be observed that Nelupure (INCI: Propanediol (and) Glycerin (and) Nymphaea caerulea Flower Extract (and) Nelumbo Nucifera Flower Extract) @ 0.2%. highly significantly inhibited (p<0.0001) Acetylcholine release.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present disclosure. Thus, it should be understood that although the present disclosure specifically disclosed by specific features, modification and variation of the concepts herein disclosed may be resorted to by those of ordinary skill in the art, and that such modifications and variations are considered to be within the scope of embodiments of the paspectention.

### Exemplary Aspects

The following exemplary aspects are provided, the numbering of which is not to be construed as designating levels of importance:

In one aspect, a method of increasing the duration of use of a topical cosmetic composition, the method comprising applying the cosmetic composition to a user followed by applying the topical cosmetic composition to the cosmetic composition.

In one aspect, the extract solution is assessed for effectiveness as an acetylcholine inhibitor

In one aspect, silica particles pre-loaded with the extract solution are used to absorb sebum and release the extract solution on sebum absorption

In one aspect, utilization of a nymphaea coerulea flower extract and nelumbo nucifera flower extract in combination with a clinically-effective amount of quercetin present to yield both antimicrobial and acetylcholine inhibitory effects.

In one aspect, increased color stability is seen as compared to a composition with an equivalent colorant load lacking the extract.

In one aspect composition has good color stability even when wetted with sweat and sebum. Color stability is suitable quantitatively measured by lack of shade change, which can be measured by color computer (SpectraFlash 600 Plus; Datacolor International Company) as delta E value of the colorimetric data before and after silicone oil was applied to the composition and wetted until max point of oil absorption. Hunter L-a-b color system is used for such colorimetric measurement.

In one aspect, the extract is utilized in a color cosmetic or skincare setting spray.

In one aspect, the use of the extract for a time and at a concentration sufficient to yield an anti-inflammatory effect when applied repeatedly to the skin.

In one aspect, the use of the extract in a topical composition that reduces pore size when applied for a time and at a concentration sufficient.

In one aspect, the use of the extract in a topical composition that yields better subsequent adherence of cosmetic formulations via decreasing skin sebum presence pre-cosmetic formula application. aspect

In one aspect, the extract is incorporated into a powder form.

In one aspect, a cosmetic primer composition comprising a effective amount of an acetylcholine release inhibitor for reducing skin inflammation and/or pore size prior to applying a color cosmetic composition.

1-12. (canceled)

13. A cosmetic method to reduce sweat comprising administering to the dermal layer of the skin where sweat glands are located a dermal composition comprising a cosmetically effective amount of an acetylcholine release inhibiting agent and a carrier, wherein the acetylcholine release inhibiting agent comprises a blend of Nelumbo nucifera and Nymphaea caerulea flower extracts.

14. The method of claim 13, wherein the blend of Nelumbo nucifera and Nymphaea caerulea flower extracts is present in an amount of from about 0.0001 wt % to about 10 wt, preferably from about 0.0005 wt % to about 5 wt %, most preferably from about 0.001 wt % to about 2 wt % based on the total weight of the composition of the total composition.

15. The method of claim 13, wherein the composition is in a cosmetically and/or dermatologically suitable form, preferably as a lotion, gel, cream, transdermal patch, in an ointment or oil base, in a sprayable liquid form or as an emulsion, a mousse, a lip balm, a lip gloss, a lotion, a mask, an ointment, a pomade, a solution, a serum, a spray, or a towelette.

16. The method of claim 15, wherein the composition is in powder form.

17. The method of claim 13, wherein the use is to treat hyperhidrosis.

18. An antiperspirant composition comprising a cosmetically effective amount of an acetylcholine release inhibiting agent and a carrier, wherein the acetylcholine release inhibiting agent comprises a blend of Nelumbo nucifera and Nymphaea caerulea flower extracts.

19. A sunscreen composition comprising a cosmetically effective amount of an acetylcholine release inhibiting agent, wherein the acetylcholine release inhibiting agent comprises a blend of Nelumbo nucifera and Nymphaea caerulea flower extracts, and a carrier.

20. A method to extend wear of a cosmetic composition while being easily removable by washing with soap and water, the method comprising:
(1) applying a composition that reduces sweat, the composition comprising a topically acceptable vehicle and a sufficient amount of an acetylcholine release inhibiting agent active ingredient comprising a blend of Nelumbo nucifera and Nymphea caerulea flower extracts.; followed by
(2) applying a cosmetic colorant composition comprising a colorant to the same skin, where the sequential application of the sebum reducing composition and the cosmetic colorant composition results in increased wear of the cosmetic colorant composition on the skin.

21. The method of claim 20, wherein the composition of step (1) is in the form of a spray.
